(19) United States
(12) Patent Application Publication
(10) Pub. No.: US 2022/0022809 A1
(43) Pub. Date: Jan. 27, 2022

(54) SYSTEMS AND METHODS TO DETECT AND TREAT OBSTRUCTIVE SLEEP APNEA AND UPPER AIRWAY OBSTRUCTION

(71) Applicants: Northwestern University, Evanston, IL (US); Ann and Robert H.Lurie Children's Hospital of Chicago, Chicago, IL (US)

(72) Inventors: Bharat Bhushan, Chicago, IL (US); Claus-Peter Richter, Skokie, IL (US); Amedee Brennan O'Gorman, Evanston, IL (US)

(21) Appl. No.: 17/309,812

(22) PCT Filed: Dec. 19, 2019
(86) PCT No.: PCT/US2019/067597

§ 371 Date: Jun. 21, 2021

Related U.S. Application Data
(60) Provisional application No. 62/781,699, filed on Dec. 19, 2018.

(51) Int. Cl. A61B 5/00; A61B 5/01; A61B 5/0205; A61B 5/024; A61B 5/053; A61B 5/08

(52) U.S. Cl. CPC A61B 5/4818; A61B 5/01; A61B 5/0205; A61B 5/024; A61B 5/6822; A61B 5/0816; A61B 5/0826; A61B 5/4809; A61B 5/4836; A61B 5/053

(57) ABSTRACT

A sleep monitor device for monitoring breathing and other physiological parameters is used to classify, assess, diagnose, and/or treat sleeping disorders (e.g., obstructive sleep apnea and upper airway obstruction, among others). The sleep monitor device can be a wearable device that contains one or more microphones arranged around the subject's neck when worn. Additionally, the wearable device may also include, or otherwise be in communication with, other sensors and/or measurement components, such as optical sources and electrodes. Using the sleep monitor device it is possible to identify upper airway resistances, the site of the obstruction, to monitor tissue resistance, temperature, and oxygen saturation. Early detection of the development of upper airway resistances during sleep can be used to control supportive measures for sleep apnea, such controlling continuous positive airway pressure (“CPAP”) devices or neurological or mechanical stimulators.

### CROSS-REFERENCE TO RELATED APPLICATIONS

This application claims the benefit of U.S. Provisional Patent Application Ser. No. 62/781,699, filed on Dec. 19, 2018, and entitled “SYSTEMS AND METHODS TO DETECT AND TREAT OBSTRUCTIVE SLEEP APNEA AND UPPER AIRWAY OBSTRUCTION.”

### BACKGROUND

Snoring, hypopnea, and apnea are characterized by frequent episodes of upper airway collapse during sleep and effects nocturnal sleep quality. Obstructive sleep apnea (“OSA”) is the most common type of sleep apnea and is caused by complete or partial cessation of breathing due to obstructions of the upper airway. It is characterized by repetitive episodes of shallow or paused breathing during sleep, despite the effort to breathe. OSA is usually associated with a reduction in blood oxygen. Individuals with OSA are rarely aware of difficulty breathing, even upon awakening. It is often recognized as a problem by others who observe the individual during episodes or is suspected because of its effects on the body. Symptoms may be present for years or even decades without identification, during which time the individual may become conditioned to the daytime sleepiness, fatigue associated with significant levels of sleep disturbances. Individuals who generally sleep alone are often unaware of the condition, without a regular bed-partner to notice and make them aware of their symptoms. As the muscle tone of the body ordinarily relaxes during sleep, and the airway at the throat is composed of walls of soft tissue, which can collapse, it is not surprising that breathing can be obstructed during sleep.

Persons with OSA have a 30% higher risk of heart attack or death than those unaffected. Over time, OSA constitutes an independent risk factor for several diseases, including systemic hypertension, cardiovascular disease, stroke, and abnormal glucose metabolism. The estimated prevalence is in the range of 3% to 7%. Sleep apnea requires expensive diagnostic and intervention paradigms, which are only available for a limited number of patients due to unavailability of sleep laboratories in each hospital. Hence, many patients with sleep apnea remain undiagnosed and untreated.

Thus, there is a need for a simple device that can enhance the diagnosis of snoring, hypopnea, and apnea such that more patients can be treated without undergoing expensive and labor-intensive full night polysomnography.

### SUMMARY OF THE DISCLOSURE

The present disclosure addresses the aforementioned drawbacks by providing a sleep monitor device that includes a support strap to be worn by a patient when sleeping having a one or more microphones coupled thereto; a processor for receiving signals from each of the one or more microphones; and a computer for receiving signals from the processor and configured to identify characteristic features from the signals and to create feature vectors for identifying different stages of normal and abnormal sleep.

It is another aspect of the disclosure to provide a method for classifying sleeping disorders in a subject. The method includes recording acoustic measurements from a neck of a subject, generating feature vectors for one or more classes of sleep by extracting feature data from the acoustic measurements using a computer system, and inputting the feature vectors to a trained machine learning algorithm, generating output as a classification of a sleep stage for the subject.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention.

### BRIEF DESCRIPTION OF THE DRAWINGS

FIG. 1 is a schematic overview of a sleep monitor device that can be implemented to classify, diagnose, monitor, and/or treat sleep disorders.

FIG. 2 is a block diagram of an example sleep monitor device according to some embodiments described in the present disclosure.

FIGS. 3A-3E show examples of sleep monitor devices according to various embodiments described in the present disclosure. FIG. 3A shows a sleep monitor device that includes a flexible support strap and a wired connection. FIG. 3B shows a sleep monitor device that includes a flexible support strap and a wireless connection unit. FIG. 3C shows a sleep monitor device that includes a rigid support strap and a wireless connection unit. FIG. 3D shows a sleep monitor device that includes a base unit that can be taped or adhered to a subject's chest. FIG. 3E shows a sleep monitor device that includes a miniaturized support and a Bluetooth connection unit.

FIG. 4 shows an example workflow diagram that depicts how a sleep monitor device may handle results from the analysis of the recorded acoustic and/or other data.

FIG. 5 shows an example workflow diagram for operating a sleep monitor device in order to generate output as a diagnosis of sleep disorder, prediction of sleep event, localization of obstruction, or control for a tactile stimulator, electrical stimulator, or CPAP device.

FIG. 6 shows an example workflow diagram of an algorithm that can be used to determine stages of breathing

FIG. 7 is a flowchart setting forth the steps of an example method for classifying, assessing, diagnosing, and/or treating sleeping disorders.

FIG. 8 illustrates an example workflow for extracting breathing rate feature data from acoustic measurement data.

FIG. 9 illustrates an example workflow for extracting frequency component feature data from acoustic measurement data.

FIG. 10 illustrates an example workflow for extracting frequency content feature data from acoustic measurement data.

FIG. 11 is a block diagram of an example system for classifying, assessing, diagnosing, and/or treating sleeping disorders in accordance with some embodiments described in the present disclosure.

FIG. 12 is a block diagram showing example components of the system for classifying, assessing, diagnosing, and/or treating sleeping disorders of FIG. 11.

### DETAILED DESCRIPTION

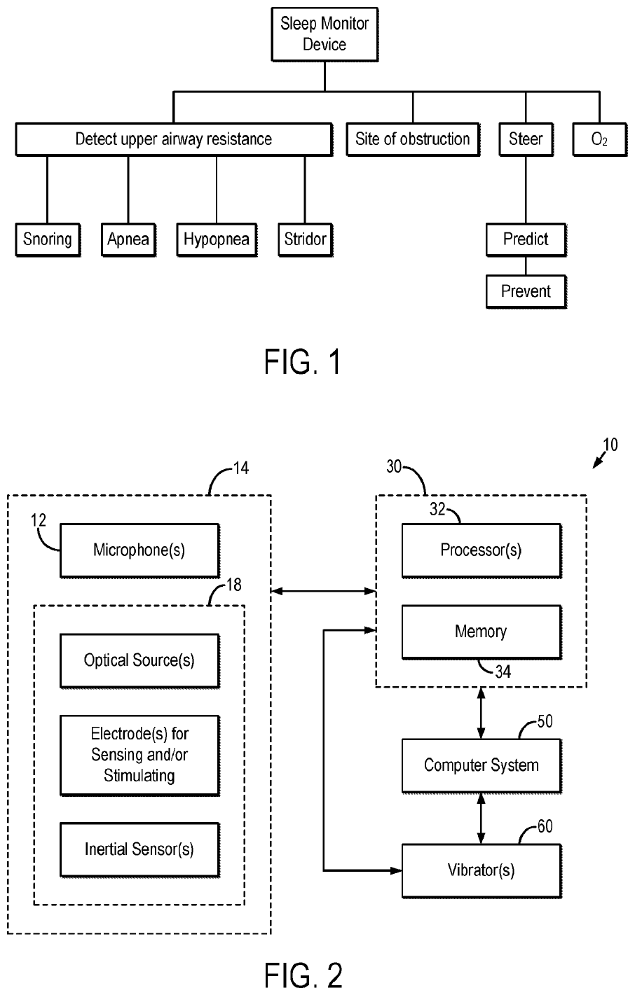

Described here are systems and methods for monitoring breathing and other physiological parameters in order to classify, assess, diagnose, and/or treat sleeping disorders (e.g., obstructive sleep apnea and upper airway obstruction, among others). In general, the systems can include a wearable device that contains one or more microphones arranged around the subject's neck. Additionally, the wearable device may also include, or otherwise be in communication with, other sensors and/or measurement components, such as optical sources and electrodes. As shown in the schematic overview of FIG. 1, with the wearable sleep monitor device it is possible to identify upper airway resistances, the site of the obstruction, to monitor tissue resistance, temperature, and oxygen saturation. Early detection of the development of upper airway resistances during sleep can be used to control supportive measures for sleep apnea, such controlling continuous positive airway pressure (“CPAP”) devices or neurological stimulators.

In some aspects, the systems and methods described in the present disclosure can recognize and identify an airway obstruction, snoring, hypopnea and apnea and to early predict its occurrence during sleep. Further, the site of the obstruction between the sternum and the pharynx can be localized. The systems and methods described in the present disclosure can also distinguish between an exhalation or inhalation stridor.

Additionally or alternatively, the systems and methods described in the present disclosure can steer from events such as snoring, hypopnea, and apnea by stimulating the individual without waking them up. In some embodiments, this may include controlling therapeutic devices, such as CPAP devices and neural stimulators. In some other embodiments, this can include controlling mechanical stimulation (e.g., vibration) provided to the subject during sleep.

As shown in FIG. 2, in one aspect of the present disclosure, a sleep monitor device 10 for classifying, assessing, diagnosing, and/or treating sleeping disorders includes one or more microphones 12 coupled to a support 14 (e.g., a neck collar, flexible strap, rigid plastic strap) to be worn by a subject in particular during night times. The sleep monitor device 10 can further include sensors/measurement components 18 for acquiring other data, such as physiological data, body position data, body motion data, or combinations thereof. In some examples, the sensors/measurement components 18 may include optical sources in the green, red, and/or infrared spectra to measure tissue temperature, heart rate, and blood oxygen saturation. Additionally or alternatively, the sensors/measurement components 18 can include one or more electrical contacts (e.g., electrodes) to measure tissue impedance, to record electrophysiological signals (e.g., electrocardiograms, electromyograms, electroencephalograms), and/or to provide electrical stimulation to the subject with electrical currents.

The microphone(s) 12 acquire acoustic measurement data (e.g., acoustic signals) that can be used to determine an acoustic fingerprint of breathing. This acoustic fingerprint can, in turn, be used to recognize and identify an airway obstruction, snoring, hypopnea, and/or apnea, and to early predict its occurrence during sleep. The acoustic fingerprint can also be used to localize the site of the obstruction between the sternum and the pharynx, and/or to distinguish between an exhalation or inhalation stridor.

In some embodiments, the sensors/measurement components 18 can include optical sources to measure oxygen saturation of the blood, determine heart rate, and/or measure the tissue temperature. Additionally or alternatively, the sensors/measurement components 18 can include one or more electrical contacts (e.g., electrodes) to measure tissue resistance, measure electrophysiology signals, or provide stimulation to steer from events such as snoring, hypopnea, and apnea by stimulating the individual without waking them up.

The sleep monitor device 10 can include a local control unit 30, which can include one or more processors 32 and a memory 34 or other data storage device or medium (e.g., an SD card or the like). In some instances, the local control unit 30 may include a base station. Signals recorded by the microphones 12 and sensors/measurement components 18 can be stored locally in the memory 34 of the sleep monitor device 10. The signal data (e.g., acoustic measurement data and/or other data) can also be filtered, amplified and digitized by the processor(s) 32 before being transferred to a computer system 50 via a wired or wireless connection. In some instances, the computer system 50 can be a hand-held device.

Alternatively, or additionally, the sleep monitor device 10 may be configured to provide mechanical stimulation, such as vibration. For instance, one or more vibrators 60 may be integrated with the support 14, or may otherwise be in communication with the local control unit 30 or computer system 50. The vibrator(s) 60 can be operable under control of the sleep monitor device 10 in order to provide mechanical stimulation to the subject, such as to steer the subject during sleep.

As will be described below, the signal data are processed with the computer system to extract characteristic features. Individual features are assembled to a feature vector, which can be used to characterize different sleep conditions. The feature data (e.g., feature vector(s)) are input to a trained machine learning algorithm to identify classified stages of normal or abnormal sleep. For example, the combined feature vector(s) from different subjects (or from prior acquisitions from the same subject) can be used to train a support vector machine (“SVM”) or other suitable machine learning algorithm, which in turn can be used to classify sleep stages from signal data acquired from the subject. The computer system 50 can also generate control instructions for controlling treatment modalities for sleep apnea, such as machines that maintain continuous positive airway pressure (“CPAP”) or electrical stimulation (e.g., neuro-stimulators). In some other instances, the computer system 50 can generate control instructions or otherwise control the operation of a mechanical stimulator, such as the vibrator(s) 60.

The control of the recording features with the sleep monitor device 10 can be implemented in a setup file for the local control unit 30 (e.g., a base station) or the computer system 50, and can be modified by the health care professional only if necessary. A toggle switch can permit visual (e.g., on-screen), standard, negative 50 V DC calibration signal for all channels to demonstrate polarity, amplitude, and time constant settings for each recorded parameter. A separate 50/60 Hz filter control can be implemented for each channel. The local control unit 30 and/or computer system 50 also enable selecting sampling rates for each channel. Additionally or alternatively, filters for data collection can functionally simulate or replicate conventional (e.g., analog-style) frequency response curves rather than removing all activity and harmonics within the specified bandwidth.

The data acquired with the sleep monitor device 10 can be retained and viewed in the manner in which they were recorded by the attending technologist (e.g., retain and display all derivation changes, sensitivity adjustments, filter settings, temporal resolution). Additionally or alternatively, the data acquired with the sleep monitor device 10 can be retained and viewed in the manner they appeared when they were scored by the scoring technologist (e.g., retain and display all derivation changes, sensitivity adjustments, filter settings, temporal resolution).

Display features settings of the sleep monitor device 10 can be controlled through software executed by the local control unit 30 and/or the computer system 50. Default settings can be implemented in a setup file and can be modified by the health care professional or examiner of the data. As one non-limiting example, the display features may include a display for scoring and review of sleep study data that meets or exceeds the following criteria: 15-inch screen-size, 1,600 pixels horizontal, and 1,050 pixels vertical. As another non-limiting example, the display features may include one or more histograms with stage, respiratory events, leg movement events, O₂ saturation, and arousals, with cursor positioning on histogram and ability to jump to the page. The display features may also include the ability to view a screen on a time scale ranging from the entire night to windows as small as 5 seconds. A graphical user interface can also be generated and provide for automatic page turning, automatic scrolling, channel-off control key or toggle, channel-invert control key or toggle, and/or change order of channel by click and drag. Display setup profiles (including colors) may be activated at any time. The display features may also include fast Fourier transformation or spectral analysis on specifiable intervals (omitting segments marked as data artifact).

The sleep monitor device 10 can also include the ability to turn off and on, as demanded, highlighting of patterns identifying respiratory events (for example apneas, hypopneas, desaturations) in a graphical user interface or other display. Additionally or alternatively, the sleep monitor device 10 can also include the ability to turn off and on, as demanded, highlighting of patterns identifying movement in a graphical user interface or other display.

Documentation and calibration procedure may be part of the device initialization. For instance, routine questions can be asked upon switching on the base station. The measurements can be compared to a set of reference data stored in the device (e.g., stored in the memory 34 or in the computer system 50). If measurements deviate more than a threshold amount (e.g., two standard deviations from the reference), the examiner can be prompted to repeat the measurement. If no reliable set of test data can be obtained, the reference values can be used for analysis of the sleep data.

In some implementations, treatment can be achieved with the sleep monitor device 10 through a conditioned reflex. A stimulus (e.g., mechanical vibration through a vibrator motor) can be conditioned to a change in breathing behavior. For example, during a one-month training period a tactile stimulus can be delivered at random times to the neck of the subject. The tactile stimulus can be given through a vibration motor, which is implemented in the sleep monitor device 10. Each time the stimulus is delivered, the subject can be asked or otherwise prompted by the sleep monitor device 10 (e.g., via a visual or auditory prompt) to take a number of deep breaths (e.g., 5 deep breaths). The number of breaths can be optimized for each subject and may, for example, be between 1 and 10. Over time, the non-specific tactile stimulus (e.g., vibration) can be conditioned, leading to a change in breathing behavior.

After the training period, the tactile stimulus can be used during the sleep stages before a subject reaches stages of hypopnea or apnea. The prediction of breathing stages (hypopnea or apnea) is done using the methods described in the present disclosure, implemented in the sleep monitor device 10. The closer the patient is to the event of hypopnea or apnea the stimulus intensity can be increased.

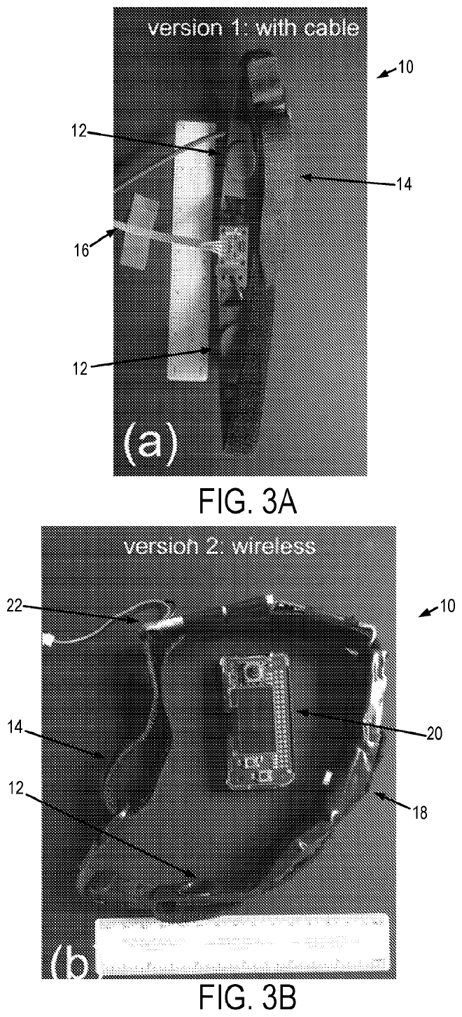

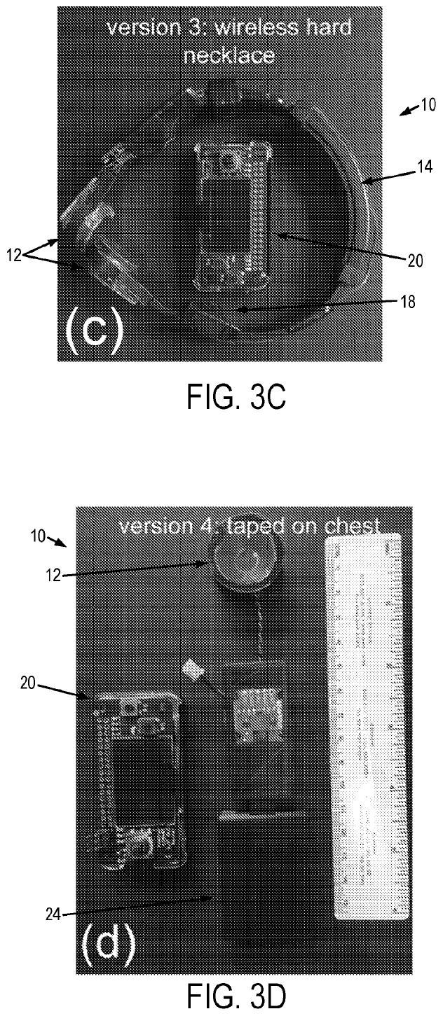

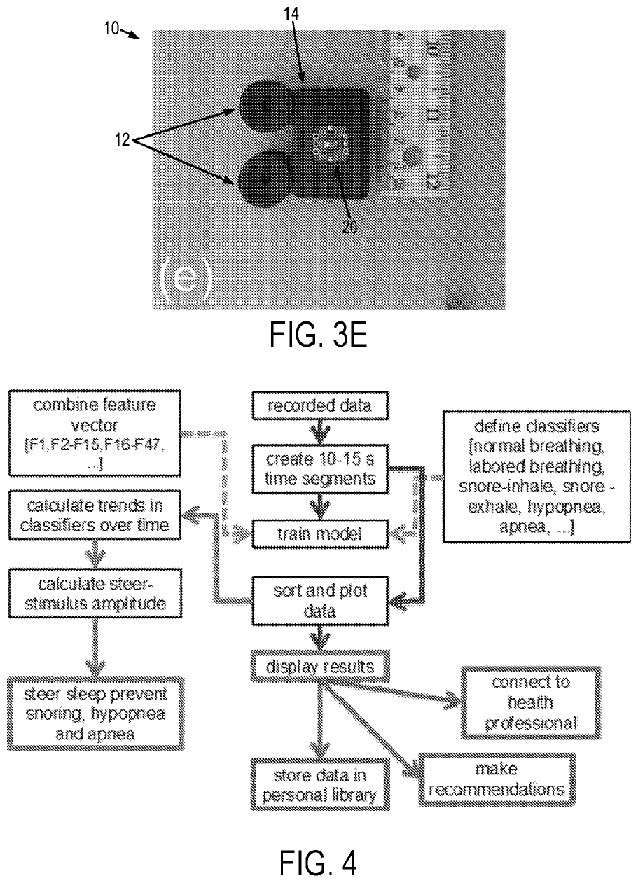

FIGS. 3A-3E show non-limiting examples of sleep monitor devices 10 in accordance with some embodiments described in the present disclosure. FIG. 3A shows an example sleep monitor device 10 that includes microphones 12 attached to a support 14, which may be constructed as a flexible strap or necklace. The microphones 12 are connected with a cable 16 from the support 14 to a computer system to record the acoustic signal of breathing during sleep.

FIGS. 3B and 3C show example sleep monitor devices 10 that, in addition to microphones 12, include other sensors/measurement components 18 such as an inertial sensor (e.g., a gyroscope) to determine body position and a pulse oximeter to measure blood oxygenation, heart rate, and tissue temperature. This example also implements wireless capability by setting up a local area network (“WLAN”) through a wireless control unit 20, which may include a programmable controller such as a Raspberry Pi. Using a wireless control unit 20 allows for recordings at any location, even in remote areas where no internet is otherwise available. The data acquired with the sleep monitor device 10 (which may include acoustic measurement data and other data, such as physiological and body position/motion data) can be stored on a local storage device (e.g., a micro SD card, a memory) and can be retrieved either directly from the local data storage device or via a secured wireless connection using the wireless control unit 20. The sleep monitor devices 10 can be powered via a battery 22 or other power source coupled to the support 14.

In the embodiment shown in FIG. 3B, the microphones 12 and other sensors/measurement components 18 are coupled to a support 14 that is constructed as a flexible strap or necklace. In the embodiment shown in FIG. 3C, the microphones 12 and other sensors/measurement components 18 are coupled to a support 14 that is constructed as a rigid housing, such as a plastic holder. A more rigid support 14 can allow for the microphones 12 and sensors/measurement components 18 to be held against the subject's skin with more consistent pressure than with a support 14 that is more flexible.

In the embodiment shown in FIG. 3D, the sleep monitor device 10 can be located remote from the subject's neck by incorporating the sensors/measurement components 18 into a housing 24 that can be taped or otherwise adhered to the subject at a location other than the neck, such as the sternum. One or more microphones 12 in electrical communication (e.g., via a wired or wireless connection) with the housing 24 can then be positioned on the subject's neck during use.

Considering the large amount of power required for the transmission of data via WLAN, in some other embodiments the wireless control unit 20 can implement a wireless connect using a Bluetooth connection between the sleep monitor device 10 and a base station. Such a configuration is shown in FIG. 3E.

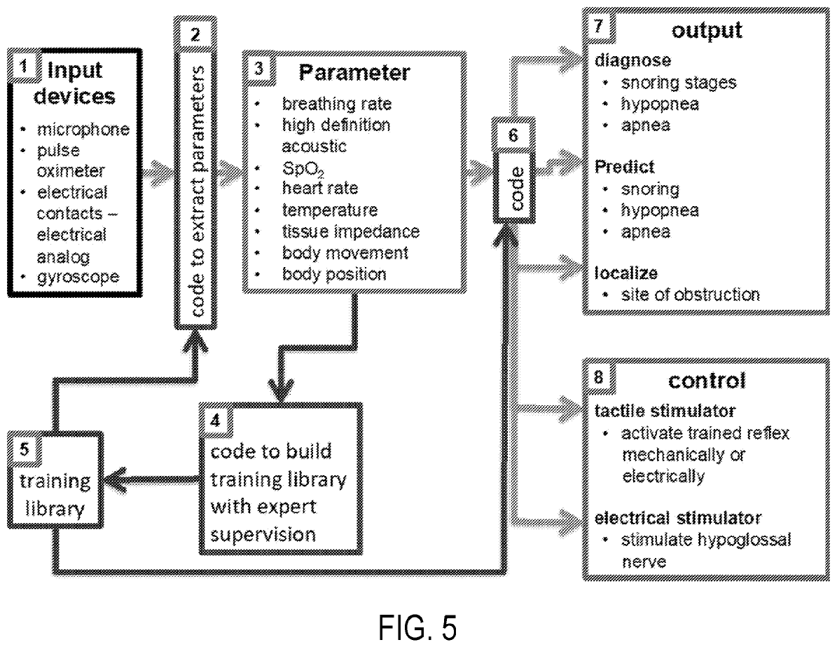

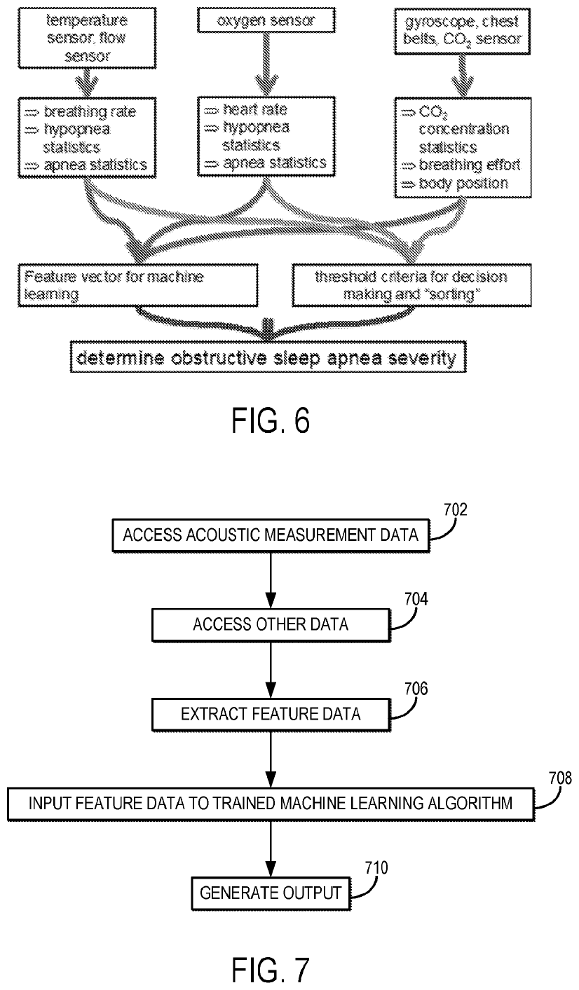

Example workflows for using the sleep monitor device described in the present disclosure are shown in FIGS. 4-6. For instance, FIG. 4 shows an example workflow diagram that depicts how a sleep monitor device may handle results from the analysis of the recorded acoustic and/or other data. FIG. 5 shows an example workflow diagram for operating a sleep monitor device in order to generate output as a diagnosis of sleep disorder, prediction of sleep event, localization of obstruction, or control for a tactile stimulator, electrical stimulator, or CPAP device. FIG. 6 shows an example workflow diagram of an algorithm that can be used to determine stages of breathing.

As described above, when using the sleep monitor device described in the present disclosure, one or more small microphones (e.g., typically but not limited to 1-10), are aligned in an array, which is secured directly on the skin over the trachea using tape or are placed on the inside of a wearable support neck collar such that they align along the trachea. The acoustic signal caused by the breathing is then captured continuously with those microphones and is transmitted (e.g., via a wired or wireless connection) to a recording device, such as but not limited to a computer, hand-held device, or single chip computer.

The recordings from the sensors may be used to determine one or more of the total sleep time, oxygen saturation, tissue temperature, sleep stages, inhalation and exhalation stridor, labored breathing, rate of breathing, wake after sleep onset, pulse rate, and tissue impedance. For instance, the signal data are subsequently analyzed and a feature vector is extracted from the acoustic signal. The analysis includes methods such as wavelet transforms, Short-Time Fourier Transforms (“STFT”), amplitude calculations, and energy calculations.

The feature vector can contain elements from the acoustic signal, breathing rate, blood oxygenation, heart rate, skin temperature, body position, and electrical fingerprints from the muscle contraction, and electrical tissue impedance. The feature vector is used to train a model (e.g., a supervised machine learning algorithm), or is otherwise input to a previously trained model. As one example, the model is used to determine different classes of breathing. The time convolution of such parameters allows the early prediction of the occurrence of a snoring event since each of the models can be tailored to an individual person. The array of microphones also allows determining the exact location of the obstruction by the acoustic fingerprint and serves as diagnostic measure for airway obstruction.

In cases when the algorithm determines that snoring/hypopnea/apnea will occur, the sleep monitor device will steer the sleeping at an early stage by stimulating the individual with electrical currents or mechanically with stimuli small enough not to wake up the person, but large enough to avoid the snoring, hypopnea, or apnea event. The stimulator can be, but not necessarily, incorporated into the collar.

Referring now to FIG. 7, a flowchart is illustrated as setting forth the steps of an example method for classifying, assessing, diagnosing, and/or treating sleeping disorders. The method includes accessing acoustic measurement data with a computer system, as indicated at step 702. The acoustic measurement data may include, for instance, acoustic signals recorded from a subject's neck. Such acoustic signals are indicative of breathing sounds that are generated by the subject during respiration. Accessing the acoustic measurement data can include retrieving previously recorded or measured data from a memory or other data storage device or medium. In some other instances, accessing the acoustic measurement data can include recording, measuring, or otherwise acquiring such data with a suitable sleep monitor device and then transferring or otherwise communicating such data to the computer system. As one non-limiting example, a sleep monitor device may include one or more microphones. For instance, the sleep monitor device may include an array of microphones, such as those described above.

In one non-limiting example, a sleep monitor device can include between 1 and 10 microphones, which may be arranged in an array when multiple microphones are used, that may be positioned such that they align along the subject's trachea. The acoustic signals caused by the breathing are then captured continuously with those microphones. The acoustic signals can be filtered, amplified, and digitized before being transmitted (e.g., via a wired or a wireless connection) to a recording device, such as but not limited to a computer system, which in some embodiments may include a hand-held device. Alternatively, the acoustic signals can be filter, amplified, and/or digitized at the computer system

The method can also include accessing other data, with the computer system, as indicated at step 704. As an example, the other data can include physiological data, such as blood oxygen saturation, body temperature, electrophysiology data (e.g., muscle activity, cardiac electrical activity), heart rate, electrical tissue impedance, or combinations thereof. Additionally or alternatively, the other data can include body position data, body movement data, or combinations thereof.

These other data can be accessed by retrieving such data from a memory or other data storage device or medium, or by acquiring such data with an appropriate measurement device or sensor and transferring the data to the computer system. The readings from the different sensors can be filtered and subsequently amplified, digitized, and continuously transmitted to the computer system, which may include a hand-held device, for further processing. Alternatively, these other data can be transferred to the computer system before filtering, amplifying, and digitizing the data.

The acoustic measurement data, other data, or both, are processed to extract feature data, as indicated at step 706. The feature data can therefore include acoustic feature data extracted from the acoustic measurement data and/or other feature data extracted from the other data. An example list of measurements and other parameters that can be included in the feature data is provided in Table 1 below. The feature data can include one or more feature vectors, which can be used to train a machine learning algorithm, or as input to an already trained machine learning algorithm, both of which will be described below in more detail.

TABLE 1
Example List of Features

Associated Sensor

General Parameters to be Measured

| | |
|---|---|
| Chin electromyogram (EMG) | Metal contacts/ |
| | electrodes |
| Airflow signals | Microphone |
| Respiratory effort signals | Microphone |
| Oxygen saturation | Optical source |
| Body position | Inertial sensor |
| Electrocardiogram (ECG) | Optical source/ECG |
| | electrode(s) |

Sleep Scoring Data

| | |
|---|---|
| Lights out clock time (hr:min) | n/a |
| Lights on clock time (hr:min) | n/a |
| Total sleep time (TST, in min) | n/a |
| Total recording time (TRT; “light out” to | n/a |
| “lights on” in min) | |
| Percent sleep efficiency (TST/TRT × 100) | n/a |

Arousal

| | |
|---|---|
| Number of arousals | Inertial sensor |
| Arousal index (ArI; number of arousals × | n/a |
| 60/TST) | |

Cardiac Events

| | |
|---|---|
| Average heart rate during sleep | Optical source |
| Highest heart rate during sleep | |
| Highest heart rate during recording | Optical source |
| Occurrence of bradycardia (if observed); | Optical source |
| report lowest heart rate | |
| Occurrence of asystole (if observed); | Optical source |
| report longest pause | |

Respiratory Events

| | |
|---|---|
| Number of obstructive apneas | Microphone |
| Number of mixed apneas | Microphone |
| Number of central apneas | Microphone |
| Number of hypopneas | Microphone |
| Number of obstructive hypopneas | Microphone |
| Number of central hypopneas | Microphone |
| Number of apneas + hypopneas | Microphone |
| Apnea index (AI; (# obstructive apneas + | n/a |
| # central apneas + # mixed apneas) × 60/TST) | |
| Hypopnea index (HI; # hypopneas × 60/ | n/a |
| TST) | |
| Apnea-Hypopnea index (AHI; (# apneas + | n/a |
| # hypopneas) × 60/TST) | |
| Obstructive apnea-hypopnea index | n/a |
| (OAHI; (# obstructive apneas + # mixed | |
| apneas + # obstructive hypopneas) × 60/TST) | |
| Central apnea-hypopnea index (CAHI; (# | n/a |
| central apneas + # central hypopneas) × | |
| 60/TST) | |
| Number of respiratory effort-related | Microphone/Inertial |
| arousals (RERAs) | sensor |
| Respiratory effort-related arousal index | Microphone/Inertial |
| (# apneas + # hypopneas + # RERAs) × 60/TST) | sensor |
| Respiratory disturbance index (RDI; (# | Microphone/Inertial |
| apneas + # hypopneas + # RERAs) × 60/TST) | sensor |
| Number of oxygen desaturations ≥3% | Optical source |
| or ≥4% | |
| Oxygen desaturation index (ODI; (# | n/a |
| oxygen desaturations ≥3% or ≥4%) × 60/TST) | |
| Arterial oxygen saturation during sleep | Optical Source |
| Minimum oxygen saturation during sleep | Optical Source |
| Occurrence of hypoventilation during | Microphone/Inertial |
| diagnostic study | sensor |

As one non-limiting example, the acoustic feature data can include breathing rate determined from the acoustic measurement data. As another non-limiting example, the acoustic feature data can include frequency components, frequency content, or both, that are extracted from the acoustic measurement data. For example, each of the traces obtained from the microphones can be fast Fourier Transformed (“FFT”), Hilbert transformed, and wavelet transformed. Hilbert transforms serve to extract the breathing rate, the FFT allows the selection of few frequency bands to calculate the variance and the energy in the selected frequency band, and the wavelet transform allows the selection of some scaling factors (frequencies) to calculate the variance and the mean of the rectified coefficients.

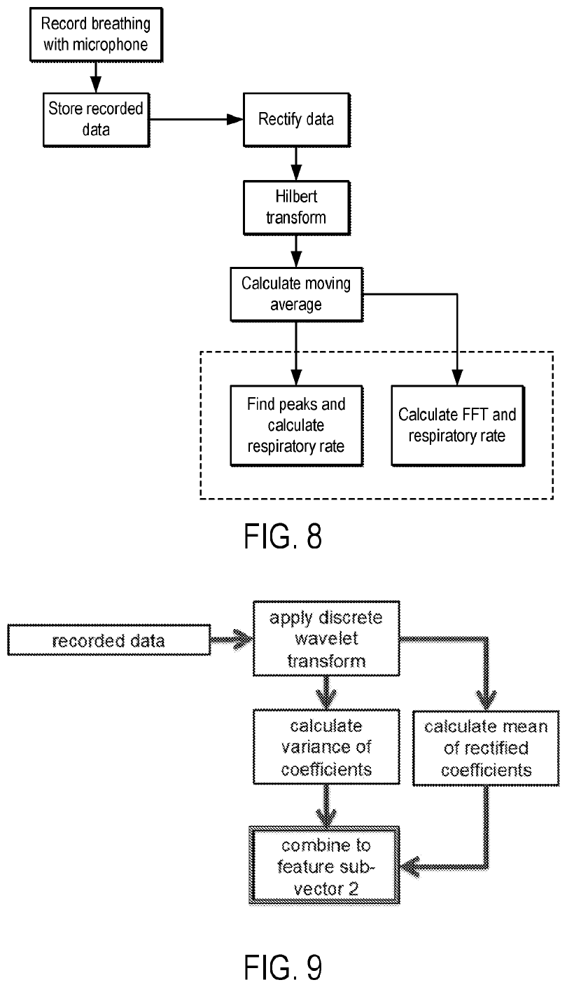

As one example, the feature data may include breathing rate. Breathing rate can be extracted from the acoustic measurement data by applying a Hilbert transform to the acoustic signals contained in the acoustic measurement data, generating output as Hilbert transformed data. In some implementations, the acoustic measurement data can be rectified before applying the Hilbert transform. As one example, peaks in the Hilbert transformed data are then identified or otherwise determined and the breathing rate is computed based on these identified peaks. As another example, a Fourier transform (e.g., a fast Fourier transform) can be applied to the Hilbert transformed data and the breathing rate can be computed from the resulting spectral data (e.g., spectrogram). In some implementations, a moving average of the Hilbert transformed data can be performed before identifying the peaks or applying the Fourier transform. An example workflow of methods for computing breathing rate from acoustic measurement data is shown in FIG. 8.

As one example, the feature data may include frequency components that can be extracted from the acoustic measurement data based on a discrete wavelet transform of acoustic signals contained in the acoustic measurement data. As shown in FIG. 9, the recording from the microphone is wavelet transformed. A number of scaling factors (which differ the most for the different classes), such as six scaling factors, are selected. The variance and the mean of the rectified coefficient are then calculated for elements of the feature vector.

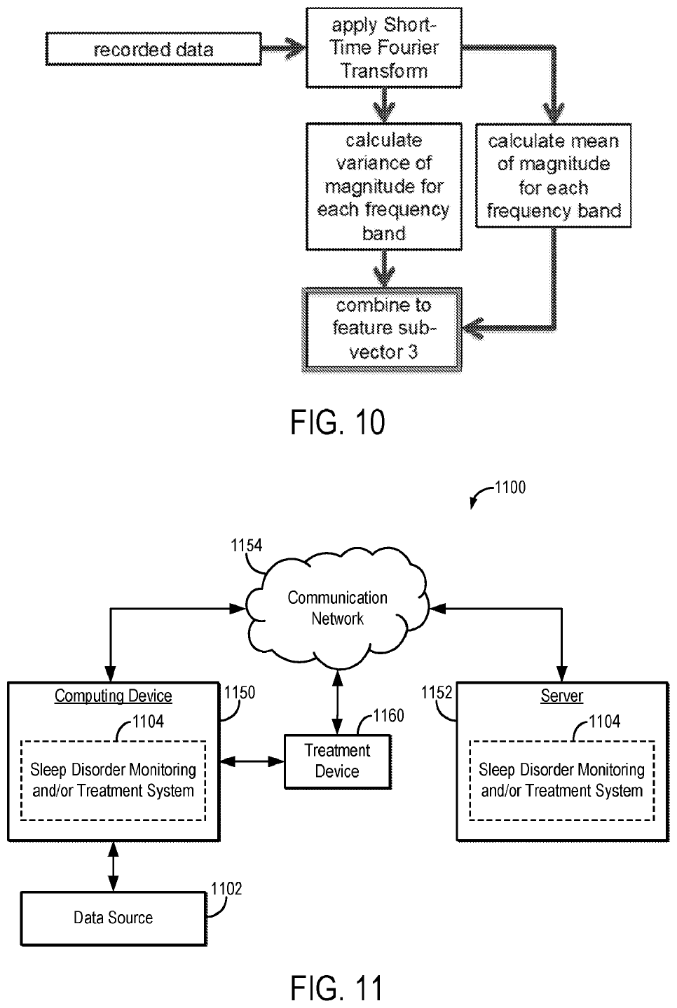

As one example, the feature data may include frequency content that can be extracted from the acoustic measurement data based on a short-time Fourier transform (“STFT”) of acoustic signals contained in the acoustic measurement data. As shown in FIG. 10, the recording from the microphone is Fast Fourier transformed. A number of scaling factors (which differ the most for the different classes), such as sixteen scaling factors, are selected. The variance and the mean of the rectified coefficients are calculated for elements of the feature vector.

As an example, the selected recording can be Short-Time-Fourier Transformed. From the resulting spectrogram, frequency bands can be selected and the average and the variation of the magnitude can be calculated and the value will be added to the feature vector. This set of elements for the feature vector originates from the frequency contents of the breathing recorded from the microphones.

As one example, the feature data may include a measurement of airflow. Airflow is used in this device to determine the rate of breathing, to characterize the sound pattern of inhalations and exhalations. Episodes of no breathing or apnea can be detected from the times between two exhales and two inhales. If the time is longer than a threshold duration (e.g., 10 seconds), an apnea event can be marked. If the breathing rate is reduced by a specified amount (e.g., 25%) of breathing rate obtained in the awake state, a hypopnea event can be marked.

As one example, the feature data may include sleep scoring data. Times when the lights are switched out and when the lights are switched on are can be recorded. From the records, the total times while the light is switched off can be calculated and stored as the total sleep time (“TST”). The ratio of total recording time can be calculated as the ratio of lights on to lights off.

As one example, the feature data may include a measure of arousal. The arousal is determined by the breathing rate and by the gyroscope readings. If the breathing rate increases above the baseline, which may be obtained while the patient is rested awake, and the gyroscope readings change, an arousal event is marked. The timing and the frequency of arousal events is stored. At the end of the study the arousal index (“Arl”) can be calculated from the number of arousals (“Nₐᵣ”) and the total sleeping time (TST) in minutes as,

$$ArI = \frac{{N}_{a r}}{TST} .$$

As one example, the feature data may include blood oxygen saturation. Blood oxygen saturation data can be obtained using a pulse oximeter, which in some embodiments may be incorporated into the sleep monitor device as described above. For instance, a pulse oximeter can be used to optically measure the pulse oxygenation (SpO₂). The fluctuation of this signal correlates with the heart rate.

As one example, the feature data may include heart rate. Heart rate data can be obtained using a pulse oximeter, a heart rate monitor, or other suitable device for measuring heart rate. In some embodiments, such devices capable of measuring heart rate may be incorporated into the sleep monitor device as described above. As one non-limiting example, heart rate can be monitored with a particle sensor that uses light sources to determine the oxygen saturation of the blood. Time segments (e.g., time segments of 10 s) can be used to determine the oxygen concentration in the blood. The readings vary with the heart and can be used to calculate the heart rate. The average heart rate and the highest heart rate during sleep and during the recording period can be continuously tracked. If the heart rate is below a threshold beats per minute, an event of bradycardia can be marked. In case the heart rate is below the threshold beats per minute, an occurrence of asystole can also be marked.

As one example, the feature data may include cardiac electrical activity that can be obtained using an electrocardiography (“ECG”) measurement device (e.g., one or more ECG electrodes), which in some embodiments may be incorporated into the sleep monitor device as described above. In some instances, heart rate can also be measured using an ECG measurement device.

As one example, the feature data may include body or skin temperature. Temperature data can be obtained using a thermometer or other temperature sensor, such as optical sources, which in some embodiments may be incorporated into the sleep monitor device as described above.

As one example, the feature data may include muscle activity measurements. Muscle activity data can be obtained using an electromyography (“EMG”) measurement device (e.g., one or more electrodes configured to measure electrical muscle activity) or the like, which in some embodiments may be incorporated into the sleep monitor device as described above. An electromyogram is a representation of the voltages, which can be measured with surface electrodes, on the skin over a muscle and which originate from the muscle activity. Sleep phases, such as the rapid eye movement (“REM”) phase can be identified in part by an increased muscle activity. For instance, muscle activity in an REM phase can be represented in an EMG recording with complexes that are larger than comparative baseline readings. In one example of the sleep monitor device described above, muscle activity data can be obtained by measuring the voltage reflecting the muscle activity using two electrodes (e.g., gold-plated electrodes, or other suitable electrodes for use in EMG) facing the skin. The electrodes may be separated by a separation distance, such as 5 mm.

As one example, the feature data may include electrical tissue impedance. Electrical tissue impedance data can be obtained using a current source and skin electrode contacts, which in some embodiments may be incorporated into the sleep monitor device as described above. As one non-limiting example, two large metal surface electrodes can be placed directly on the skin. An alternating current of 1 Hz to 40 Hz at 0 mA to 1 mA can be passed between the electrode contacts for short time periods, typically not longer than 5 s. The corresponding driving voltage is recorded and the resistance calculated as the ratio of the measured voltage and the driving current. In between tissue impedance measurements, which may occur every minute, the electrode contacts can be used to measure the electrical activity produced by the muscles below (i.e., to record muscle activity data as EMG data). The variation and mean energy can be calculated form the recorded traces.

As one example, the feature data may include body position and/or motion measurements. Body position data can be obtained using one or more inertial sensors, which in some embodiments may be incorporated into the sleep monitor device as described above. As an example, an inertial sensor can include one or more accelerometers, one or more gyroscopes, one or more magnetometers, or combinations thereof. The baseline measures of the inertial sensor can determine the orientation of the front section of the neck-band. Large spikes in the traces recorded with the inertial sensor(s) will indicate the presence of body movements. The movement can be scaled according to the maximum amplitude-peak in the inertial sensor readings.

Referring again to FIG. 7, the feature data are input to a trained machine learning algorithm, as indicated at step 708, generating output as indicated at step 710. In some implementations, feature data obtained from the subject can be used to train the machine learning algorithm, such that the trained machine learning algorithm is a subject-specific implementation. In other instances, the machine learning algorithm can be trained on feature data from other subjects, which are stored as training data in a training library or database.

As one non-limiting example, the machine learning algorithm can be a support vector machine (“SVM”). In other embodiments, other machine learning algorithms or models may also be trained and implemented.

As described above, in some implementations inputting the feature data to the trained machine learning algorithm generates output as a classification and/or diagnosis of a sleeping disorder, a sleeping stage, or the like. Each feature vector can represent one stage of sleeping or a class. A machine learning model can be trained and optimized for each individual subject using previously extracted feature vectors (i.e., training data that includes feature data extracted from other subjects). As one non-limiting example, according to the feature data, the classes defined can include normal breathing, snoring, exhalation stridor, inhalation stridor, normal breathing rate, hypopnea, and apnea.

For various sleeping stages or classes, a characteristic reading for this stage is captured from each sensor and combined into a multidimensional feature vector. The vector is then used by a model to recognize sleep stages automatically. Classification can then be used to determine trends during the sleep cycles and to early predict snoring, hypopnea, and/or apnea.

As described above, in some implementations inputting the feature data to the trained machine learning algorithm generates output as a prediction of a sleep event, such as snoring, hypopnea, and/or apnea. For instance, the change of the feature vector over time allows the early prediction of an event. This trend can be used for an early intervention in treating hypopnea or apnea.

As described above, in some implementations inputting the feature data to the trained machine learning algorithm generates output as a localization of where an obstruction is within the subject's anatomy.

As described above, in some implementations inputting the feature data to the trained machine learning algorithm generates output as a control instructions or parameters for controlling a treatment device, such as a tactile stimulator, an electrical stimulator, and/or a CPAP device. Intervention (such as low level electrical or mechanical stimulation that would not disturb the patient's sleep phases but still evoke an acquired reflex) can be steered to optimize treatment and decrease effects on the patient.

In some implementations, the feature data can be stored as training data and used to train a machine learning algorithm. For a selected group of patients, the data can be analyzed by sleep expert. During the analysis, the clinician can determine at which time during the night hypopnea, apnea, or snoring occurs. The expert can also characterize the breathing sounds regarding exhalation or inhalation stridor. After the expert has labeled a given condition, the file can be copied automatically into a similarly named training library. During the training process of the machine learning algorithm, all files in the training library can be utilized for training. The structure of the training library allows for expansion in the future because each category can easily be resorted.

The training library can be composed of multiple sets of recordings that are sorted and labeled for the different sleep conditions as determined by experts in the field from the polysomnography, which can be obtained in parallel to the stored data sets. If required, the training library can be expanded, checked, refined, or relabeled.

Referring now to FIG. 11, an example of a system 1100 for classifying, assessing, diagnosing, and/or treating sleeping disorders in accordance with some embodiments of the systems and methods described in the present disclosure is shown. As shown in FIG. 11, a computing device 1150 can receive one or more types of data (e.g., acoustic measurement data, physiological data, body position data, body motion data, or other data) from data source 1102, which may be an acoustic measurement or other data source. In some embodiments, computing device 1150 can execute at least a portion of a sleep disorder monitoring and/or treatment system 1104 to classify, assess, diagnose, and/or treat sleeping disorders from data received from the data source 1102.

Additionally or alternatively, in some embodiments, the computing device 1150 can communicate information about data received from the data source 1102 to a server 1152 over a communication network 1154, which can execute at least a portion of the sleep disorder monitoring and/or treatment system. In such embodiments, the server 1152 can return information to the computing device 1150 (and/or any other suitable computing device) indicative of an output of the sleep disorder monitoring and/or treatment system 1104.

In some embodiments, computing device 1150 and/or server 1152 can be any suitable computing device or combination of devices, such as a desktop computer, a laptop computer, a smartphone, a tablet computer, a wearable computer, a server computer, a virtual machine being executed by a physical computing device, and so on. As one non-limiting example, the computing device 1150 can be integrated with the sleep monitor device 10. As another non-limiting example, the computing device 1150 can include a base station that is in communication with the sleep monitor device. As still another non-limiting example, the computing device 1150 can include a computer system or hand-held device that is in communication with the base station.

In some embodiments, data source 1102 can be any suitable source of acoustic measurement and/or other data (e.g., physiological data, body position/motion data), such as microphones, optical sources, electrodes, inertial sensors, another computing device (e.g., a server storing data), and so on. In some embodiments, data source 1102 can be local to computing device 1150. For example, data source 1102 can be incorporated with computing device 1150 (e.g., computing device 1150 can be configured as part of a device for capturing, scanning, and/or storing images). As another example, data source 1102 can be connected to computing device 1150 by a cable, a direct wireless link, and so on. Additionally or alternatively, in some embodiments, data source 1102 can be located locally and/or remotely from computing device 1150, and can communicate data to computing device 1150 (and/or server 1152) via a communication network (e.g., communication network 1154).

In some embodiments, a treatment device 1160 can be in communication with the computing device 1150 and/or server 1152 via the communication network 1154. As an example, control instructions generated by the computing device 1150 can be transmitted to the treatment device 1160 to control a treatment delivered to the subject. The treatment device 1160 may be a CPAP machine. In other implementations, the treatment device 1160 may be electrodes for providing electrical stimulation, which may include neurostimulation. Such electrodes may, in some configurations, be integrated into the sleep monitor device 10.

In some embodiments, communication network 1154 can be any suitable communication network or combination of communication networks. For example, communication network 1154 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), a wired network, and so on. In some embodiments, communication network 1154 can be a local area network, a wide area network, a public network (e.g., the Internet), a private or semi-private network (e.g., a corporate or university intranet), any other suitable type of network, or any suitable combination of networks. Communications links shown in FIG. 11 can each be any suitable communications link or combination of communications links, such as wired links, fiber optic links, Wi-Fi links, Bluetooth links, cellular links, and so on.

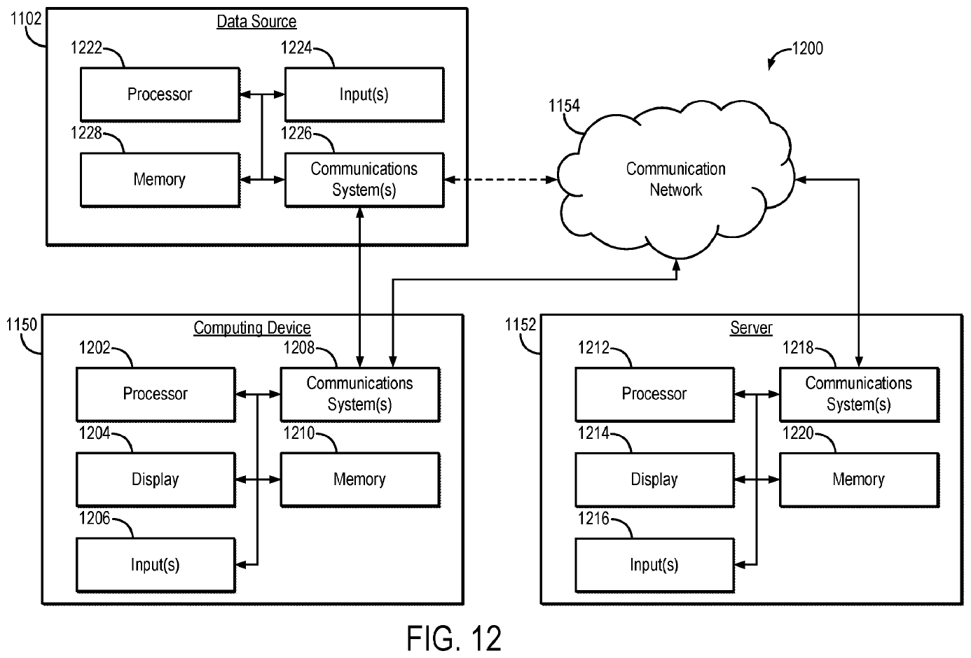

Referring now to FIG. 12, an example of hardware 1200 that can be used to implement data source 1102, computing device 1150, and server 1152 in accordance with some embodiments of the systems and methods described in the present disclosure is shown. As shown in FIG. 12, in some embodiments, computing device 1150 can include a processor 1202, a display 1204, one or more inputs 1206, one or more communication systems 1208, and/or memory 1210. In some embodiments, processor 1202 can be any suitable hardware processor or combination of processors, such as a central processing unit (“CPU”), a graphics processing unit (“GPU”), and so on. In some embodiments, display 1204 can include any suitable display devices, such as a computer monitor, a touchscreen, a television, and so on. In some embodiments, inputs 1206 can include any suitable input devices and/or sensors that can be used to receive user input, such as a keyboard, a mouse, a touchscreen, a microphone, and so on.

In some embodiments, communications systems 1208 can include any suitable hardware, firmware, and/or software for communicating information over communication network 1154 and/or any other suitable communication networks. For example, communications systems 1208 can include one or more transceivers, one or more communication chips and/or chip sets, and so on. In a more particular example, communications systems 1208 can include hardware, firmware and/or software that can be used to establish a Wi-Fi connection, a Bluetooth connection, a cellular connection, an Ethernet connection, and so on.

In some embodiments, memory 1210 can include any suitable storage device or devices that can be used to store instructions, values, data, or the like, that can be used, for example, by processor 1202 to present content using display 1204, to communicate with server 1152 via communications system(s) 1208, and so on. Memory 1210 can include any suitable volatile memory, non-volatile memory, storage, or any suitable combination thereof. For example, memory 1210 can include RAM, ROM, EEPROM, one or more flash drives, one or more hard disks, one or more solid state drives, one or more optical drives, and so on. In some embodiments, memory 1210 can have encoded thereon, or otherwise stored therein, a computer program for controlling operation of computing device 1150. In such embodiments, processor 1202 can execute at least a portion of the computer program to present content (e.g., images, user interfaces, graphics, tables), receive content from server 1152, transmit information to server 1152, and so on.

In some embodiments, server 1152 can include a processor 1212, a display 1214, one or more inputs 1216, one or more communications systems 1218, and/or memory 1220. In some embodiments, processor 1212 can be any suitable hardware processor or combination of processors, such as a CPU, a GPU, and so on. In some embodiments, display 1214 can include any suitable display devices, such as a computer monitor, a touchscreen, a television, and so on. In some embodiments, inputs 1216 can include any suitable input devices and/or sensors that can be used to receive user input, such as a keyboard, a mouse, a touchscreen, a microphone, and so on.

In some embodiments, communications systems 1218 can include any suitable hardware, firmware, and/or software for communicating information over communication network 1154 and/or any other suitable communication networks. For example, communications systems 1218 can include one or more transceivers, one or more communication chips and/or chip sets, and so on. In a more particular example, communications systems 1218 can include hardware, firmware and/or software that can be used to establish a Wi-Fi connection, a Bluetooth connection, a cellular connection, an Ethernet connection, and so on.

In some embodiments, memory 1220 can include any suitable storage device or devices that can be used to store instructions, values, data, or the like, that can be used, for example, by processor 1212 to present content using display 1214, to communicate with one or more computing devices 1150, and so on. Memory 1220 can include any suitable volatile memory, non-volatile memory, storage, or any suitable combination thereof. For example, memory 1220 can include RAM, ROM, EEPROM, one or more flash drives, one or more hard disks, one or more solid state drives, one or more optical drives, and so on. In some embodiments, memory 1220 can have encoded thereon a server program for controlling operation of server 1152. In such embodiments, processor 1212 can execute at least a portion of the server program to transmit information and/or content (e.g., data, images, a user interface) to one or more computing devices 1150, receive information and/or content from one or more computing devices 1150, receive instructions from one or more devices (e.g., a personal computer, a laptop computer, a tablet computer, a smartphone), and so on.

In some embodiments, data source 1102 can include a processor 1222, one or more inputs 1224, one or more communications systems 1226, and/or memory 1228. In some embodiments, processor 1222 can be any suitable hardware processor or combination of processors, such as a CPU, a GPU, and so on. In some embodiments, the one or more input(s) 1224 are generally configured to acquire data, and can include one or more microphones, one or more optical sources, one or more electrodes, one or more inertial sensors, and so on. Additionally or alternatively, in some embodiments, one or more input(s) 1224 can include any suitable hardware, firmware, and/or software for coupling to and/or controlling operations of microphones, optical sources, electrodes, and/or inertial sensors. In some embodiments, one or more portions of the one or more input(s) 1224 can be removable and/or replaceable.

Note that, although not shown, data source 1102 can include any suitable inputs and/or outputs. For example, data source 1102 can include input devices and/or sensors that can be used to receive user input, such as a keyboard, a mouse, a touchscreen, a microphone, a trackpad, a trackball, and so on. As another example, data source 1102 can include any suitable display devices, such as a computer monitor, a touchscreen, a television, etc., one or more speakers, and so on.

In some embodiments, communications systems 1226 can include any suitable hardware, firmware, and/or software for communicating information to computing device 1150 (and, in some embodiments, over communication network 1154 and/or any other suitable communication networks). For example, communications systems 1226 can include one or more transceivers, one or more communication chips and/or chip sets, and so on. In a more particular example, communications systems 1226 can include hardware, firmware and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 1228 can include any suitable storage device or devices that can be used to store instructions, values, data, or the like, that can be used, for example, by processor 1222 to control the one or more input(s) 1224, and/or receive data from the one or more input(s) 1224; to images from data; present content (e.g., images, a user interface) using a display; communicate with one or more computing devices 1150; and so on. Memory 1228 can include any suitable volatile memory, non-volatile memory, storage, or any suitable combination thereof. For example, memory 1228 can include RAM, ROM, EEPROM, one or more flash drives, one or more hard disks, one or more solid state drives, one or more optical drives, and so on. In some embodiments, memory 1228 can have encoded thereon, or otherwise stored therein, a program for controlling operation of data source 1102. In such embodiments, processor 1222 can execute at least a portion of the program to generate images, transmit information and/or content (e.g., data, images) to one or more computing devices 1150, receive information and/or content from one or more computing devices 1150, receive instructions from one or more devices (e.g., a personal computer, a laptop computer, a tablet computer, a smartphone, etc.), and so on.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (e.g., hard disks, floppy disks), optical media (e.g., compact discs, digital video discs, Blu-ray discs), semiconductor media (e.g., random access memory (“RAM”), flash memory, electrically programmable read only memory (“EPROM”), electrically erasable programmable read only memory (“EEPROM”)), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

The present disclosure has described one or more preferred embodiments, and it should be appreciated that many equivalents, alternatives, variations, and modifications, aside from those expressly stated, are possible and within the scope of the invention.

1. A sleep monitor device, comprising:
a support strap to be worn by a patient when sleeping having a one or more microphones coupled thereto;
a processor for receiving signals from each of the one or more microphones; and
a computer for receiving signals from the processor and configured to identify characteristic features from the signals and to create feature vectors for identifying different stages of normal and abnormal sleep.

2. The sleep monitor device of claim 1, further comprising one or more sensors for measuring one or more of tissue temperature, heart rate, and blood oxygen saturation of the patient, and for transmitting signals from the one or more sensors to the processor; the processor further being configured to transmit the signals from the one or more sensors to the computer; and the computer further configured to correlate the signals from the one or more sensors with the signals from the one or more microphones when creating the feature vectors.

3. The sleep monitor device of claim 2, further comprising one or more electrical contacts for accomplishing one or more of measuring tissue impedance, measuring an electrophysiology signal, and providing stimulation to the patient upon the detection of an abnormal sleep condition.

4. The sleep monitor device of claim 1, further comprising one or more electrical contacts for accomplishing one or more of measuring tissue impedance, measuring an electrophysiology signal, and providing stimulation to the patient upon the detection of an abnormal sleep condition.

5. The sleep monitor device of claim 3 or 4, wherein the one or more electrical contacts for providing stimulation to the patient comprise one or more of an electrode for delivering electrical current to the patient.

6. The sleep monitor device of any one of claims 1-4, further comprising one or more vibrators for providing mechanical stimulation to the patient upon the detection of an abnormal sleep condition.

7. The sleep monitor device of any one of claims 1-4, wherein the computer is configured to determine one or more of total sleep time, oxygen saturation, tissue temperature, sleep stage, inhalation and exhalation stridor, labored breathing, rate of breathing, wake after sleep onset, heart rate, and tissue impedance based on the signals received by the computer from the processor.

8. The sleep monitor device of any one of claims 1-4, wherein the one or more microphones are located on the support strap so as to be aligned with the patient's trachea when the support strap is worn by the patient.

9. The sleep monitor device of any one of claims 1-4, wherein the support strap is a flexible support strap.

10. The sleep monitor device of any one of claims 1-4, wherein the support strap comprises a rigid support.

11. A method for classifying sleeping disorders in a subject, comprising:
(a) recording acoustic measurements from a neck of a subject;
(b) generating feature vectors for one or more classes of sleep by extracting feature data from the acoustic measurements using a computer system;
(c) inputting the feature vectors to a trained machine learning algorithm, generating output as a classification of a sleep stage for the subject.

12. The method of claim 11, further comprising delivering stimulation to the subject upon determination that the subject is in an abnormal sleep stage.

13. The method of claim 12, wherein the stimulation comprises one of mechanical stimulation or electrical stimulation.

14. The method of claim 11, further comprising controlling a continuous positive airway pressure to adjust a pressure setting upon determination that the subject is in an abnormal sleep stage.

15. The method of claim 11, wherein the trained machine learning algorithm comprises a support vector machine.

16. The method of claim 11, wherein the classes of sleep comprise one or more of normal breathing, snoring, exhalation stridor, inhalation stridor, normal breathing rate, hypopnea, and apnea.

17. The method of claim 11, further comprising recording physiological data from the subject with one or more sensors, and wherein generating the feature vectors for one or more classes of sleep also comprises extracting feature data from the physiological data.

18. The method of claim 17, wherein the physiological data comprises at least one of oxygen saturation data, heart rate data, electrophysiology data, body position data, electrical tissue impedance data, temperature data, or body movement data.

19. The method of claim 11, wherein the feature data comprise at least one of breathing rate, frequency components of the acoustic measurements, or frequency content of the acoustic measurements.

20. The method of claim 11, further comprising localizing an airway obstruction in the subject based on output generated by inputting the feature vectors to the trained machine learning algorithm.
